(19) United States
(12) Patent Application Publication
(10) Pub. No.: US 2018/0199853 A1
(43) Pub. Date: Jul. 19, 2018

(54) MRI MACHINE FOR MEASURING AT LEAST A PORTION OF A HEAD

(71) Applicant: DENTSPLY SIRONA Inc., York, PA (US)

(72) Inventors: Ciamak ABKAI, Heddesheim (DE); Tilman NIEDERFÜHR, Weinheim (DE); Klaus STÖCKL, Bensheim (DE); Guido SCHEIBL, Bickenbach (DE); Johannes ULRICI, Darmstadt (DE)

(73) Assignee: DENTSPLY SIRONA Inc., York, PA (US)

(21) Appl. No.: 15/869,787

(22) Filed: Jan. 12, 2018

(30) Foreign Application Priority Data
Jan. 13, 2017 (EP) 17151488.8

(51) Int. Cl. A61B 5/055; G01R 33/30

(52) U.S. Cl. CPC A61B 5/0555; G01R 33/307

(57) ABSTRACT

Disclosed herein is an MRI machine for measuring at least a portion of a head of a patient, having at least one primary magnetic field unit and at least one patient opening. The MRI machine has a patient seat, where the patient seat has adjusting means, which are driven by at least one drive means controlled by a control unit to move the patient seat into the patient opening along a travel curve as far as an imaging position, in which the head of the patient is arranged at least partially in an imaging area of the MRI machine, wherein an adjustable element is attached to the patient seat relative to the patient seat and wherein, by means of a detector apparatus, the position of the adjustable element relative to the primary magnetic field unit and/or relative to the patient seat is automatically determined on the MRI machine.

### TECHNICAL FIELD

The present disclosure relates to an MRI machine for measuring at least a portion of a head of a patient, comprising at least one primary magnetic field unit and at least one patient opening.

### BACKGROUND

The prior art discloses several MRI machines for measuring a patient.

DE 10 2009 027119 B4 discloses an MRI system for imaging a head area with a permanent magnet, a gradient coil and at least one high-frequency coil, wherein a magnetic field unit may be adjusted in height on a vertically-arranged stand. In addition, the magnetic field unit may be pivoted up to an angle of 45° relative to the longitudinal axis of the stand. The patient takes a seated position during the measurement, with the patient's head positioned relative to the stand by a forehead support and earpieces.

In this MRI system, the patient is positioned in a complex manner relative to the MRI machine, in which the patient is positioned via a cylindrical patient opening. In this case, a height-adjustable stool and the vertically-adjustable stand of the MRI system are manually adjusted to allow the patient's head to be measured.

DE 197 34 138 B2 discloses an MRI machine comprising a gradient coil assembly and an MRI main coil.

US 2013/0023418 A1 discloses an MRI machine with a cooling system consisting of a first stage and a second stage.

The object of the present disclosure is thus to provide an MRI machine which allows convenient and simple positioning of the patient relative to the MRI machine.

### SUMMARY

An MRI machine for measuring at least a portion of a head of a patient, comprising at least one primary magnetic field unit and at least one patient opening, wherein the MRI machine has a patient seat, wherein the patient seat has adjusting means, driven by at least one drive means and controlled by means of a control unit, in order to move the patient seat into the patient opening along a travel curve as far as an imaging position, at which the head of the patient lies at least partially in an imaging area of the MRI machine, wherein an adjustable element is attached to the patient seat relative to the patient seat, and wherein, by means of a detector apparatus on the MRI machine, a position of the adjustable element relative to the primary magnetic field unit and/or relative to the patient seat is automatically determined.

### BRIEF DESCRIPTION OF THE DRAWINGS

The disclosure is explained with reference to the drawings. The drawings show:

FIG. 1 a sketch of an MRI machine,

FIG. 2 the adjustment of the headrest,

FIG. 3 a sketch to illustrate the adjustment of the high-frequency coil.

### DETAILED DESCRIPTION

The present disclosure relates to an MRI machine for measuring at least a portion of a head of a patient, comprising at least one primary magnetic field unit and at least one patient opening. The MRI machine has a patient seat, wherein the patient seat has adjusting means driven by at least one drive means controlled by a control unit to guide the patient seat into the patient opening along a travel curve until it reaches an imaging position, in which the head of the patient is at least partially arranged in an imaging area of the MRI machine. An adjustable element is attached to the patient seat relative to the patient seat, wherein by means of a detector apparatus on the MRI machine, a position of the adjustable element relative to the primary magnetic field unit and/or relative to the patient seat is determined automatically.

The MRI machine (magnetic resonance imaging machine) is a conventional MRI machine for measuring a head. In this case, the MRI machine has a measurement volume, wherein the object volume of the object is arranged within the measurement volume in order to measure the object, i.e. the portion of the head.

The primary magnetic field unit generates the primary magnetic field and may, for example, consist of at least one permanent magnet, at least one magnetic coil, or at least one superconducting magnetic coil.

The patient opening may be of any shape, for example, a circular shape, an elliptical shape or a rectangular shape. The patient opening must be designed to be sufficiently large so that, in particular, the patient's head area fits into the measuring area of the MRI machine.

The portion of the head to be imaged may, for example, comprise a lower jaw, an upper jaw and/or the TMJ.

The patient seat may include a backrest and a seat surface, wherein the patient seat is arranged on the MRI machine or has a known position relative to the MRI machine. An adjusting means of the patient seat may be, for example, a guide rail which guides the patient seat along the travel curve relative to the MRI machine. The drive means may be, for example, an electric motor, a hydraulic drive or a pneumatic drive. The control unit may be, for example, a microcomputer or a microchip, wherein the control unit controls the drive means so that the patient seat is adjusted as required. When positioning the patient, the patient seat is moved into the patient opening along the travel curve up to the imaging position. The imaging position is determined by the portion of the head. This is because in the imaging position, the portion of the head to be imaged is arranged within the imaging area of the MRI machine. The adjustable element is arranged on the patient seat and may be manually adjusted by a user or driven by second adjusting means and a second drive means. The adjustable element is positioned relative to the patient prior to moving the patient in. The detector apparatus is arranged on the MRI machine and makes it possible to determine the position of the adjustable element relative to the primary magnetic field unit and/or relative to the patient seat.

An advantage of the present MRI machine is that the patient already positioned on the patient seat may be moved fully automatically into the MRI machine in a convenient manner. The automatic determination of the position of the adjustable element indirectly determines the position of the head of the patient and thus of the portion of the head to be imaged relative to the primary magnetic field unit and/or relative to the patient seat, so that the length of the travel curve still to be traveled to the imaging position may be determined, wherein the portion of the head to be imaged is arranged in the imaging area of the MRI machine.

Another advantage of the present MRI machine is that positioning errors are prevented by determining the position prior to entry, or upon entry, and by automatically positioning the patient instead of manual positioning of the patient.

Advantageously, the adjustable element may be a patient positioning element, in particular an adjustable chinrest, an adjustable headrest, adjustable earpieces, adjustable headphones and/or an element to be adjusted with respect to the positioned patient, in particular an adjustable high-frequency coil.

The adjustable element may be adjusted, for example, using a second adjusting means, such as a guide rail, relative to the patient seat. The chinrest, the headrest, the earpieces, the headphones and/or the high-frequency coil may, for example, be manually adjusted relative to the patient chair along such a guide rail by a user or by a drive means. The adjustable element may be adjusted as a function of the position of the head relative to the patient seat, i.e. the size of the patient. Subsequently, the patient's chair is moved into the MRI machine.

Advantageously, the detector apparatus may determine the position of the adjustable element relative to the primary magnetic field unit and/or relative to the patient seat prior to moving in the patient seat, upon moving in the patient seat or only upon reaching the imaging position.

When determining the position of the adjustable element before moving in the patient, therefore, the length of the travel curve still to be traversed may be determined. When determining the position upon moving in the patient, the length of the remaining distance before reaching the imaging position may be determined. In the third alternative, the position is determined upon reaching the imaging position, wherein the drive means is stopped and the moving in of the patient seat is stopped.

Advantageously, the axis of travel may have an angle relative to a central axis of the MRI machine which lies within an angular range tolerance between +10° and −10° relative to a central axis of the MRI machine.

The central axis of the MRI machine may, for example, coincide with an axis of symmetry of the patient opening. The central axis of the MRI machine may also coincide with the axis of symmetry of the primary magnetic field of the primary magnetic field unit, or may be aligned parallel to the axis of symmetry of the primary magnetic field.

By aligning the travel curve or the adjusting means, such as a guide rail, parallel to the central axis of the MRI machine, convenient moving in of the patient seat into the patient opening is made possible.

Advantageously, the detector apparatus may be a laser apparatus based on a triangulation measurement method, a phase shift method or a transit time method, wherein the laser apparatus has a laser, a reflector and a sensor, wherein the laser is at a defined position relative to the MRI machine, and wherein the reflector is arranged on the element.

A precise determination of the position of the adjustable element is made possible by use of the laser apparatus.

Advantageously, the detector apparatus may be a cable pull apparatus comprising a cable pull potentiometer and a cable eye, wherein the cable pull potentiometer has a defined position relative to the MRI machine and the cable eye is arranged on the element.

By using the cable pull apparatus, a reliable determination of the position of the adjustable element is made possible.

Advantageously, the detector apparatus may have a sensor, such as a light barrier, and a switching flag, wherein the sensor has a defined position relative to the MRI machine and the switching flag is arranged on the adjustable element, wherein the sensor is arranged such that upon moving in the patient seat, or upon reaching the imaging position, the sensor generates a signal as soon as the switching flag passes the sensor.

Reliable determination of the position of the adjustable element when moving in the patient seat is made possible by using the light barrier.

Advantageously, the MRI machine may have a selection apparatus, wherein the patient's imaging position to be adjusted relative to the imaging area of the MRI machine is automatically determined with computer assistance or by a user by means of the selection apparatus as a function of the anatomical structure of the patient to be imaged, a fixed application and/or patient-specific information.

The selection apparatus may be embodied, for example, as a selection menu with operating elements, such as mechanical buttons or virtual buttons on a touchscreen. The selection apparatus allows, for example, the selection of an anatomical structure to be imaged, a specific application and/or patient-specific information. The anatomical structure to be imaged may be, for example, a lower jaw, an upper jaw and/or the TMJ. The fixed application may be, for example, a virtual panoramic scan, a cephalometric scan, or a 3D volume scan of a particular area. The patient-specific information may relate, for example, to the position of the portion of the head to be imaged.

The disclosure further relates to a method for positioning the head of a patient relative to an MRI machine, comprising at least one primary magnetic field unit and at least one patient opening. In this case, a patient seat is attached to the MRI machine, wherein the patient seat has adjusting means driven by at least one drive means controlled by means of a control unit in order to move the patient seat into the patient opening along a travel curve as far as an imaging position at which the head of the patient is at least partially arranged in an imaging area of the MRI machine, wherein an adjustable element that is adjustable relative to the patient seat is attached to the patient seat. The position of the adjustable element relative to the primary magnetic field unit and/or relative to the patient seat is automatically determined by means of a detector apparatus on the MRI machine, wherein the position of the adjustable element is used to determine a length of the travel curve of the patient seat for the control of the control unit.

In the present method of positioning, the above-described MRI machine is used to drive the patient into the imaging position fully automatically. The specific position of the adjustable element is used to determine the length of the travel curve to be traveled until the imaging position is reached.

An advantage of this method is that the patient is automatically moved into the imaging position in a convenient and precise manner. As a result, compared to the manual positioning of the patient, positioning errors are prevented.

Advantageously, the imaging position of the patient to be adjusted relative to the imaging area of the MRI machine may be automatically determined with computer assistance or by a user by means of a selection apparatus as a function of the anatomical structure of the patient to be imaged, a fixed application and/or patient-specific information.

By means of the selection apparatus, therefore, the anatomical structure to be imaged, the specific application, or the patient-specific information is selected. Depending on this selection, the position of the imaging position is then determined relative to the imaging area of the MRI machine. The selection apparatus may be arranged on the MRI machine or also externally outside the MRI machine, for example in the form of a PC with a monitor and operating elements, such as a mouse and a keyboard.

Advantageously, in a first step, the patient may be positioned on the patient seat, wherein, in a second step, the adjustable element is adjusted with respect to an imaged anatomical structure of the patient, wherein, in a third step, the patient positioned on the patient seat uses the adjusting means to be moved into the imaging position.

This sequence of the individual method steps ensures precise positioning of the patient.

Advantageously, the drive means may be switched off automatically when the specific imaging position is reached.

By switching off the drive means by means of the control unit, the moving in of the patient seat is therefore stopped.

Advantageously, the detector apparatus may determine the position of the adjustable element relative to the MRI machine prior to moving in the patient seat, upon moving in the patient seat, or only upon reaching the imaging position.

In the first alternative, the position is determined prior to moving in the patient seat so that the length of the travel curve still to be traveled may be determined. In the second alternative, the position is determined upon moving in the patient seat so that remaining travel may be determined until the imaging position is reached. In the third alternative, the position is determined only upon reaching the imaging position, whereupon the drive is then turned off and the moving stopped.

### DESIGN EXAMPLES

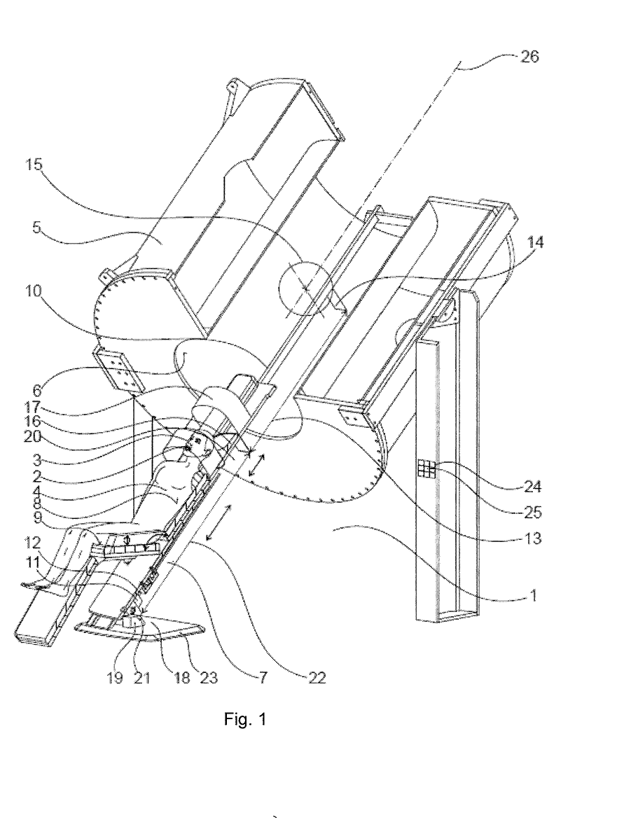

FIG. 1 shows a sketch of an MRI machine 1 for measuring at least one portion 2 of a head 3 of a patient 4, comprising at least one primary magnetic field unit 5 and at least one patient opening 6. The MRI machine 1 furthermore has a patient seat 7, which has a backrest 8 and a seating surface 9. In addition, the patient seat 7 has an adjusting means 10, such as a guide rail, and a drive means 11, such as an electric motor, to drive the patient seat into the patient opening 6. The drive means 11 is controlled by means of a control unit 12 to move the patient seat 7 in the patient opening 6 along a travel curve 13 (shown as an arrow) to an imaging position 14, in which the portion 2 of the head 3 is arranged in an imaging area 15 of the MRI machine. The imaging area 15 of the MRI machine 1 is represented by a ball in the sketch. The portion 2 to be imaged is indicated by a cross in the sketch. In the imaging position 14, the portion 2 to be imaged is arranged within the imaging area 15 of the MRI machine 1. An adjustable element, such as a headrest 16 and/or a high-frequency coil 17 is arranged on the patient seat 7, and may be adjusted manually or driven by a drive means along an adjustment axis by means of a second adjusting means, such as a guide rail, relative to the patient seat 7, as indicated by the arrow. The headrest 16 may be adjusted together with the high-frequency coil 17 or independently of the high-frequency coil 17. By means of a detector apparatus 18, which is designed in the present case as a laser apparatus and has a laser 19, a reflector 20 and a sensor 21, the laser light of the laser 19 is reflected by the reflector 20 and detected by the sensor 21 to determine the distance 22 between the laser 19 and the reflector 20. The reflector 20 is attached to the adjustable element, such as the head rest 16 and/or on the high-frequency coil, so that the position of the adjustable element relative to the laser 19 and thus relative to the patient seat 7 and/or relative to the primary field unit 5 may be determined. The laser 19 is arranged at a defined position relative to the primary field unit 5 on a stand 23. The detector apparatus 18 may also be embodied as a cable pull apparatus consisting of a cable pull positioner and a cable eye on the adjustable element or as a light barrier with a switching flag on the adjustable element 16, 17. The switching flag may be arranged at any defined position relative to the primary magnetic field unit 5. On the MRI machine 1, a selection apparatus 24 is implemented with selection elements 25, such as buttons or virtual operating elements on a touchscreen. The selection apparatus 24 allows the selection of an anatomical structure to be imaged, a specific application and/or patient-specific information. The imaging position 14 is indicated by the position of the reflector 20 upon the moving in of the patient seat 7 along the travel curve 13, wherein the patient and patient seat 7 in the imaging position 14 are not illustrated for reasons of clarity. The axis of travel 13, which is determined by the rail 10, is arranged parallel, or at least within a tolerance range between +10° and −10°, relative to a central axis 26 of the MRI machine 1. In the present case, the central axis 26 coincides with an axis of symmetry of the patient opening 6 and with an axis of symmetry of a primary magnetic field of the primary magnetic field unit 5.

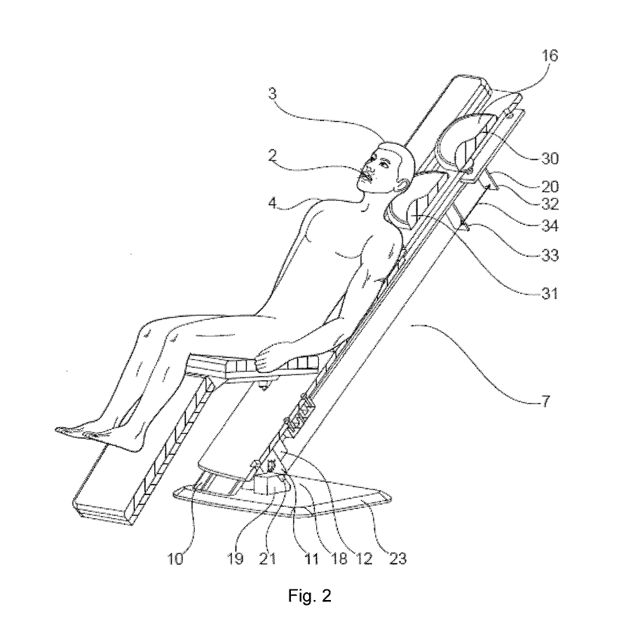

FIG. 2 shows the adjustment of the headrest 16 relative to the patient seat 7, beginning at a starting position 30 to an end position 31, wherein the headrest 16 is arranged below the head 3 of the patient 4. During the displacement of the headrest 16, the reflector 20 is also moved from a starting position 32 to an end position 33, as indicated by the arrow 34. The displacement of the headrest 16 may, for example, take place by means of a second adjusting means, such as a guide rail (not shown).

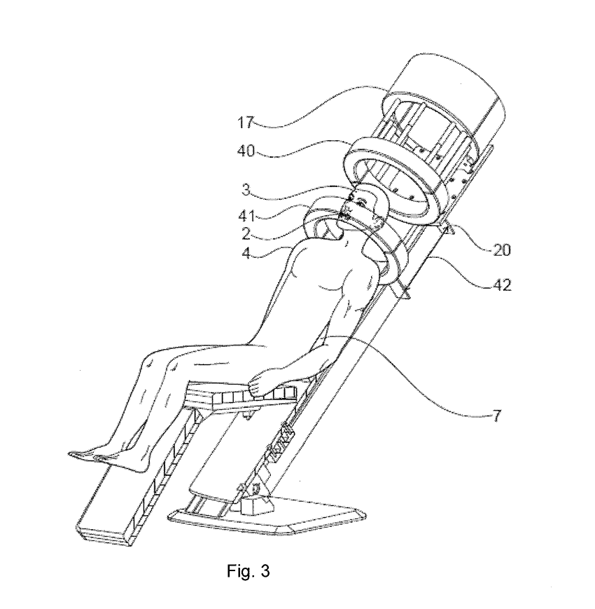

FIG. 3 shows a sketch to illustrate the adjustment of the high-frequency coil 17 relative to the patient seat 7, starting from a starting position 40 to an end position 41, as indicated by the arrow 42. The high-frequency coil 17 is arranged only through the lower ring at the end position 41, so that the portion 2 of the head 3 of the patient 4 to be imaged is arranged within the high-frequency coil.

After the adjustment of the adjustable element, such as the headrest 16 of FIG. 2 or the high-frequency coil 17 of FIG. 3, the patient seat 7 is moved into the patient opening 6 to the imaging position 14, as shown in FIG. 1.

### REFERENCE SIGNS

1 MRI machine
2 portion
3 head
4 patient
5 primary magnetic field unit
6 patient opening
7 patient seat
8 backrest
9 seat surface
10 adjusting means
11 drive means
12 control unit
13 travel curve
14 imaging position
15 imaging area
16 headrest
17 high-frequency coil
18 detector apparatus
19 laser
20 reflector
21 sensor
22 distance
23 stand
24 selector apparatus
25 selection elements
26 central axis
30 starting position
31 end position
32 starting position
33 end position
34 arrow
40 starting position
41 end position
42 arrow

1. An MRI machine for measuring at least a portion of a head of a patient, comprising at least one primary magnetic field unit and at least one patient opening,
wherein the MRI machine has a patient seat,
wherein the patient seat has adjusting means, driven by at least one drive means and controlled by means of a control unit, in order to move the patient seat into the patient opening along a travel curve as far as an imaging position,
at which the head of the patient lies at least partially in an imaging area of the MRI machine,
wherein an adjustable element is attached to the patient seat relative to the patient seat, and
wherein, by means of a detector apparatus on the MRI machine,
a position of the adjustable element relative to the primary magnetic field unit and/or relative to the patient seat is automatically determined.

2. The MRI machine according to claim 1, wherein the adjustable element is a patient positioning element.

3. The MRI machine according to claim 1, wherein the detector apparatus determines the position of the adjustable element relative to the primary magnetic field unit and/or relative to the patient seat before moving in the patient seat, upon moving in the patient seat or only upon reaching the imaging position.

4. The MRI machine according to claim 1, wherein the MRI machine has a selection apparatus, wherein the imaging position to be adjusted of the patient relative to the imaging area of the MRI machine is automatically determined with computer assistance or by a user by means of the selection apparatus as a function of the anatomical structure of the patient to be imaged, a fixed application and/or patient-specific information.

5. A method for positioning a head (of a patient relative to an MRI machine, comprising at least one primary magnetic field unit and at least one patient opening,
wherein a patient seat is attached to the MRI machine,
wherein the patient seat has adjusting means driven by at least one drive means and controlled by means of a control unit in order to move the patient seat into the patient opening along a travel curve to an imaging position at which the head of the patient is arranged at least partially in an imaging area of the MRI machine,
wherein an adjustable element is attached to the patient seat relative to the patient seat,
wherein, by means of a detector apparatus (18) on the MRI machine, the position of the adjustable element relative to the primary magnetic field unit and/or relative to the patient seat is determined automatically, and
wherein the position of the adjustable element is used to determine a length of the travel curve of the patient seat for the control of the control unit.

6. The method according to claim 5, wherein the imaging position be adjusted of the patient relative to the imaging area of the MRI machine is automatically determined with computer assistance or manually by a user by means of a selection apparatus as a function of an anatomical structure of the patient to be imaged, a fixed application and/or patient-specific information.

7. The method according to claim 5, wherein in a first step the patient is positioned on the patient seat, wherein in a second step, the adjustable element is adjusted with respect to an anatomical structure of the patient to be imaged, while in a third step, the patient positioned on the patient seat is moved into the imaging position by means of the adjusting means.

8. The method according to claim 5, wherein upon reaching the predetermined imaging position, the drive means is automatically switched off.

9. The method according to claim 5, wherein the detector apparatus determines the position of the adjustable element relative to the MRI machine before moving in the patient seat, upon moving in the patient seat or only upon reaching the imaging position.
